(19) United States
(12) Patent Application Publication
(10) Pub. No.: US 2020/0360592 A1
(43) Pub. Date: Nov. 19, 2020

(54) DIALYSIS CATHETER, IN PARTICULAR FOR LONG-TERM USE

(71) Applicant: Joline GmbH & Co. KG, Hechingen (DE)

(72) Inventor: Dieter SEIDENBERGER, Hechingen (DE)

(21) Appl. No.: 16/638,914

(22) PCT Filed: Jun. 15, 2018
(86) PCT No.: PCT/EP2018/065922

§ 371 Date: Feb. 13, 2020

(30) Foreign Application Priority Data
Aug. 17, 2017 (DE) 10 2017 118 819.3

(51) Int. Cl. A61M 1/36; A61M 25/00; A61M 1/14

(52) U.S. Cl. CPC A61M 1/3661; A61M 25/003; A61M 2025/0073; A61M 2025/0031; A61M 2025/0037; A61M 1/14

(57) ABSTRACT

The invention relates to a catheter for dialysis, in particular for long-term use,
having a proximal end for insertion into a blood vessel,
having a middle wall extending along a longitudinal axis and forming a midplane, wherein the middle wall separates an introduction lumen from a removal lumen,
wherein the removal lumen has at the proximal end a removal opening for the removal of blood, and the introduction lumen has at the proximal end an introduction opening for the introduction of purified blood, wherein
the removal opening is located in a plane which intersects the midplane in a section line at an acute angle w₁,
wherein the section line and the longitudinal axis enclose an acute angle α, and
in that the introduction opening is located in a plane which intersects the midplane in a section line s₂ at an acute angle,
wherein the section line and the section line enclose an angle which lies in the range from 60° to 120°.

### BACKGROUND OF THE INVENTION

### 1. Field of the Invention

The invention relates to a dialysis catheter, in particular for long-term use, having a proximal end for insertion into a blood vessel, having a distal end for connection to a dialyzer, having an outer wall with a cross-section with a round contour, having a middle wall extending along a longitudinal axis and forming a midplane E_{w}, wherein the middle wall separates an introduction lumen from a removal lumen, and wherein the removal lumen has at the proximal end a removal opening for the removal of blood, and the introduction lumen has at the proximal end an introduction opening for the introduction of purified blood. In the following, the term dialysis shall refer to all blood purification processes, in particular hemodialysis, hemofiltration, hemodiafiltration, peritoneal dialysis, hemoperfusion, and apheresis processes.

Such a catheter can particularly be used for a longer period of time, several days, weeks or months. The catheter is preferably made of a plastic and further preferably made of polyurethane.

### 2. Description of Related Art

Such catheters are known in a variety of styles from the prior art. For example, EP 2 574 365 A1 and EP 2 923 722 A1 disclose such catheters, each of which has a middle wall that separates the introduction lumen from the removal lumen.

In order to ensure a suitable removal of blood and a suitable introduction of purified blood, the proximal ends of the known catheters have different geometries. In particular, it must be ensured that a suction of the introduction opening on a vessel wall is prevented as much as possible. In order to prevent recirculation, it must also be ensured that the introduced blood is not removed again through the removal opening.

WO 2013/163172 A1 discloses catheters which have expandable lumens at the proximal end. US 2015/0088100 A1 discloses catheters which, on the side, have spindle-shaped openings at the proximal end.

### SUMMARY OF THE INVENTION

The present invention therefore addresses the problem of proposing a dialysis catheter which has a suitable proximal end.

This problem is solved in that the removal opening is located in a plane E₁ which intersects the midplane E_{w} in a section line s₁ at an acute angle w₁, wherein the section line s₁ and the longitudinal axis l enclose an acute angle α. It is further provided that the introduction opening is located in a plane E₂ which intersects the midplane E_{w} in a section line s₂ at an acute angle w₂, wherein the section line s₁ and the section line s₂ enclose an angle β which lies in the range from 60° to 120°.

The fact that both the removal opening and the introduction opening are not perpendicular to the middle wall, but instead each enclose an acute angle w₁ and w₂ with the middle wall, and that the section lines s₁ and s₂ themselves enclose an angle β, blood can be removed in a suitable manner and purified blood can be introduced into the blood vessel. If the angle β lies particularly in the range from 80° to 100° and further preferably in the range of 90°, favorable conditions are achieved.

The angle α can preferably be in the range from 30° to 60° and preferably further in the range of 45°. This also results in favorable flow conditions.

Furthermore, it can be provided that the angles w₁ and w₂ are designed to be identical. In such case, the angles can lie in the range from 30° to 60° and further preferably in the range of 45°.

The configuration can be such that the intersection point S of the section line s₁ and the section line s₂ lies outside the middle wall. This results in a suitable distance between the section line s₁ and the section line s₂, once again creating favorable conditions.

The proximal end of the catheter preferably has a free end edge, which is formed by the middle wall and is located in an end line. Said end line preferably runs parallel to the section line s₂ or encloses an angle with the section line s₂ which lies in the range from 0° to 15°. In the event that the angle is 0°, the end line lies in the section line s₂. This also results in favorable flow conditions.

The middle wall as such preferably has edges running parallel to the longitudinal axis l, wherein the end edge encloses an acute angle w₄ with one edge and an obtuse angle w₅ with the other edge. The end edge thus runs obliquely to the longitudinal axis l.

The angle w₄ can preferably lie in the range from 30° to 60° and further preferably in the range of 45°. Since the edges run parallel to each other, the obtuse angle w₅ is of an amount that is 180° minus the amount of w₄.

It has proven advantageous if the introduction lumen has at least one additional opening arranged at a distance from the introduction opening. It has also proven advantageous if the removal lumen has at least one additional opening arranged at a distance from the removal opening. Blood can additionally be introduced or removed through the additional openings. This is particularly necessary if one of the openings comes to bear against a blood vessel and is covered by the blood vessel.

The cross-section of the introduction lumen advantageously corresponds to the cross-section of the removal lumen. The two lumens can be designed to be mirror-symmetrical to the middle wall.

The catheter according to the invention preferably has two lumens. However, it is conceivable that a third or fourth lumen can also be provided, for example, in order to introduce medication or irrigation fluids into the blood vessel.

Further details and advantageous embodiments of the invention can be found in the following description, on the basis of which one embodiment of the invention shall be described and explained in more detail.

### BRIEF DESCRIPTION OF THE DRAWING

In the drawings:

FIG. 1 shows the side view of the proximal end of the catheter with the removal opening;

FIG. 2 shows the view according to FIG. 1 rotated by 180° about the longitudinal axis l;

FIG. 3 shows a cross-section of the catheter according to FIG. 1 along the line III;

FIG. 4 shows the bottom view of the proximal end of the catheter according to FIG. 1;

FIG. 5 shows the top view of the proximal end of the catheter according to FIG. 1; and

FIG. 6 shows a perspective view of the proximal end of the catheter with the removal opening.

### DETAILED DESCRIPTION OF THE BEST MODE OF THE INVENTION

The drawings show a dialysis catheter 10 having a proximal end 12 for insertion into a blood vessel. The catheter 10 has a middle wall 14 extending along a longitudinal axis l and forming a midplane E_{w}.

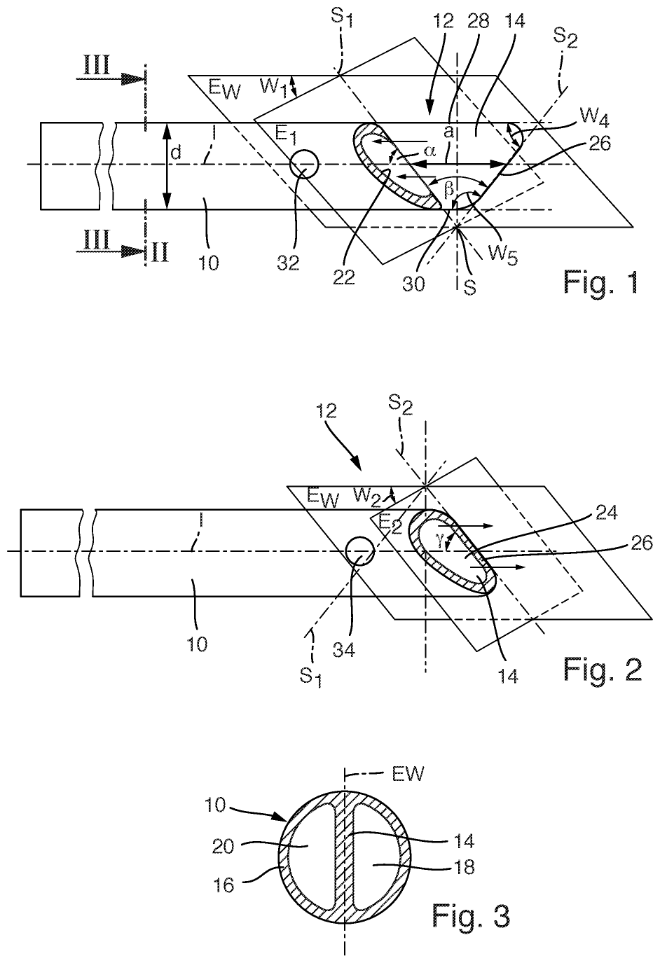

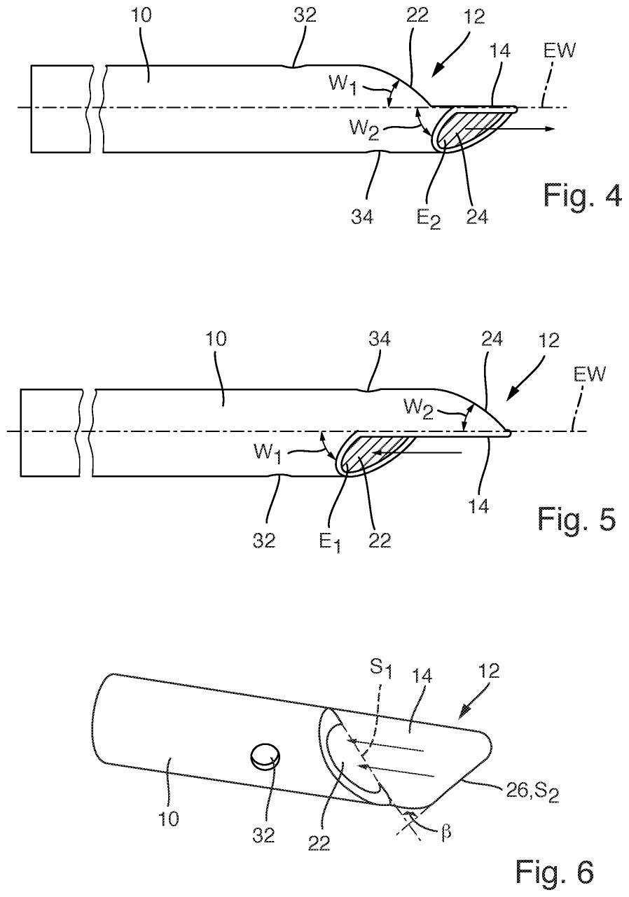

As can be seen in FIG. 3, the cross-section of the catheter has a round contour with an outer wall 16. The middle wall 16 separates the inside of the catheter into an introduction lumen 18 and a removal lumen 20. At the proximal end 12, the removal lumen 20 has a removal opening 22. In this case, the removal opening 22 is located in a plane E₁ indicated in FIG. 1.

At the proximal end 12, the introduction lumen 20 has an introduction opening 24. As is apparent from FIG. 2, the introduction opening 24 is located in a plane E₂.

As shown in FIG. 1, the plane E₁ encloses an acute angle w₁ with the midplane E_{w}. In this case, the angle w₁ is approximately 45°. As is also apparent from FIG. 1, the removal opening 22 is arranged at a distance from the free end of the catheter which is formed by a free end edge 26 of the middle wall 14. The distance a, measured along the longitudinal axis l, essentially corresponds to the diameter d of the outer wall 16.

As is apparent from FIG. 2, the midplane E_{w} and the plane E₂ enclose the angle w₂, wherein the angle w₂ lies in the range of 45°. As is also apparent from FIG. 2, the end edge 26 is located in the section line s₂ of the plane E_{w} in the plane E₂. The section line s₂ intersects the longitudinal axis l at the angle γ. The angle γ is preferably 45°.

As is apparent from FIG. 1, the plane E₁ intersects the plane E_{w} in the section line s₁. The section line s₁ intersects the longitudinal axis l at the angle α. The angle α is preferably 45°. As is also apparent from FIG. 1, the section line s₁ intersects the section line S₂, which runs along the end edge 26, at an angle β. The angle β preferably lies in the range from 80° to 90°.

It is also apparent from FIG. 1 that the intersection point S of the section line s₁ with the section line s₂ lies outside the middle wall 14.

It is also apparent from FIG. 1 that, in the region of the proximal end 14, on the side proximal to the removal opening 22, the middle wall 14 has two edges 28, 30 running parallel to the longitudinal axis. The edge 28 encloses an acute angle w₄ of 45° with the end edge 26 or the section line s₂. The other edge 30 encloses an obtuse angle w₅ in the range of 135° with the end edge 26 or the section line s₁.

As is particularly apparent from FIGS. 1 and 2, the introduction lumen 20 has an additional opening 32 spaced apart from the removal opening 22. This additional opening 32 serves to ensure that blood can also be removed in case the introduction opening 22 is closed, for example, by bearing against a blood vessel. Correspondingly, as is apparent from FIG. 2, an additional opening 34 is provided on the introduction lumen 18.

The described geometric configuration of the proximal end 12 of the catheter 10 has the advantage that, during the operation of the catheter 10, blood can be optimally removed and purified blood can be optimally introduced into a blood vessel.

1. Catheter (10) for dialysis, in particular for long-term use, having a proximal end (12) for insertion into a blood vessel, having a middle wall (14) extending along a longitudinal axis l and forming a midplane E_{w}, wherein the middle wall (14) separates an introduction lumen (18) from a removal lumen (20), and wherein the removal lumen (20) has at the proximal end (12) a removal opening (22) for the removal of blood, and the introduction lumen (18) has at the proximal end (12) an introduction opening (24) for the introduction of purified blood, characterized in that
the removal opening (22) is located in a plane E₁ which intersects the midplane E_{w} in a section line s₁ at an acute angle w₁, wherein the section line s₁ and the longitudinal axis l enclose an acute angle α, and that the introduction opening (24) is located in a plane E₂ which intersects the midplane E_{w} in a section line s₂ at an acute angle w₂, wherein the section line s₁ and the section line s₂ enclose an angle β which lies in the range from 60° to 120°.

2. Catheter (10) according to claim 1, characterized in that the angle β lies in the range from 80° to 100°, and further lies in the range of 90°.

3. Catheter (10) according to claim 1, characterized in that the acute angle α lies in the range from 30° to 60°, and further in the range of 45°.

4. Catheter (10) according to claim 1, characterized in that angles w₁ and w₂ are of the same size and/or lie in the range from 30° to 60°, and further in the range of 45°.

5. Catheter (10) according to claim 1, characterized in that the intersection point S of the section line s₁ and the section line s₂ lies outside the middle wall (14).

6. Catheter (10) according to claim 1, characterized in that the proximal end (12) has a free end edge (26) which is located in an end line, wherein the end line runs parallel to the section line s₂ or encloses an angle with the section line s₂ which lies in the range from 0° to 15°.

7. Catheter (10) according to claim 6, characterized in that the free end edge (26) is formed by the middle wall (14).

8. Catheter (10) according to claim 6, characterized in that the middle wall (14) has two edges (28, 30) running parallel to the longitudinal axis l, wherein the free end edge (26) encloses an acute angle w₄ with one edge (28) and an obtuse angle w₅ with another edge (30).

9. Catheter (10) according to claim 8, characterized in that the acute angle w₄ lies in the range from 30° to 60° and further preferably in the range of 45°.

10. Catheter (10) according to claim 1, characterized in that the introduction lumen (18) has at least one additional opening arranged at a distance from the introduction opening (24).

11. Catheter (10) according to claim 1, characterized in that the removal lumen (20) has at least one additional opening (32) arranged at a distance from the removal opening (22).

12. Catheter (10) according to claim 1, characterized in that the introduction lumen (20) has a cross-section that corresponds to a corresponding cross-section of the removal lumen (20).

13. Catheter (10) according to claim 2, characterized in that the acute angle α lies in the range from 30° to 60°, and further in the range of 45°.

14. Catheter (10) according to claim 2, characterized in that angles w₁ and w₂ are of the same size and/or lie in the range from 30° to 60°, and further in the range of 45°.

15. Catheter (10) according to claim 3, characterized in that angles w₁ and w₂ are of the same size and/or lie in the range from 30° to 60°, and further in the range of 45°.

16. Catheter (10) according to claim 2, characterized in that the intersection point S of the section line s₁ and the section line s₂ lies outside the middle wall (14).

17. Catheter (10) according to claim 3, characterized in that the intersection point S of the section line s₁ and the section line s₂ lies outside the middle wall (14).

18. Catheter (10) according to claim 4, characterized in that the intersection point S of the section line s₁ and the section line s₂ lies outside the middle wall (14).

19. Catheter (10) according to claim 2, characterized in that the proximal end (12) has a free end edge (26) which is located in an end line, wherein the end line runs parallel to the section line s₁ or encloses an angle with the section line s₁, which lies in the range from 0° to 15°.

20. Catheter (10) according to claim 3, characterized in that the proximal end (12) has a free end edge (26) which is located in an end line, wherein the end line runs parallel to the section line s₁ or encloses an angle with the section line s₁, which lies in the range from 0° to 15°.
